(19) United States
(12) Patent Application Publication
(10) Pub. No.: US 2013/0041211 A1
(43) Pub. Date: Feb. 14, 2013

(54) Desensitising Apparatus

(71) Applicant: Antonios Camille Zamar, Eastbourne (GB)

(72) Inventor: Antonios Camille Zamar, Eastbourne (GB)

(73) Assignee: AURIS MEDICAL LIMITED, Eastbourne, Sussex (GB)

(21) Appl. No.: 13/643,158

(22) PCT Filed: Apr. 28, 2011
(86) PCT No.: PCT/GB11/50847

§ 371 Date: Oct. 24, 2012

(30) Foreign Application Priority Data
Apr. 29, 2010 (GB) 1007153.8

(51) Int. Cl. A61F 5/00; A61H 1/00

(52) U.S. Cl. 600/38; 601/46

(57) ABSTRACT

The present invention encompasses a vibration unit (7) for use in penis desensitising apparatus (11), the vibration unit comprising a main body (9) and one or more side elements (10) projecting outwardly therefrom, the vibration unit thereby having an exterior profile for promoting anchoring thereof within the desensitising apparatus. Further there is provided a system which affords the vibration unit (7) with a plurality of desensitising apparatuses, and a method for using the desensitising apparatus, where the apparatus is pivoted in and out of contact with the user's penis.

The present invention relates to desensitising apparatus for use in stimulating body parts, in particular the penis.

In this regard, the applicant of the present invention has previously disclosed apparatus of this sort in EP1049433. In EP1049433, the applicant identified a method of alleviating the problems associated with premature ejaculation, this method involving the targeted stimulation of the penis to an extent whereby it becomes desensitized and tolerized to other stimulants that may lead to ejaculation. As a result, the latency period to ejaculation can be delayed.

According to the present invention there is provided a vibration unit for use in penis desensitising apparatus, the vibration unit comprising:—a main body and one or more side elements projecting outwardly therefrom, the vibration unit thereby having an exterior profile for promoting anchoring thereof within the desensitising apparatus. This arrangement provides a secure housing of the vibration unit when in use and allows removal of the vibration unit from a desensitising apparatus when necessary, for example when necessary for replacement, cleaning, repair or upgrade.

Preferably, the vibration unit main body is substantially elongate, with the one or more side elements extending substantially normally therefrom, and is removably housed within said desensitising apparatus, which itself is formed of a flexible resilient material. The side elements afford transverse rigidity to the apparatus allowing a user to grip the apparatus firmly in use.

Conveniently, the desensitising apparatus is formed with a deformable aperture which in its undeformed state matches at least part of the exterior profile of the vibration unit.

Preferably, the vibration unit comprises a motor, a driven axle, and an asymmetrically mounted vibrator, with the motor and driven axle being mounted along the longitudinal axis of the main body. This provides for a compact arrangement. Alternatively, the vibration unit comprises a “pancake” motor.

Switching components can moreover be housed within the one or more side elements which are disposed normally to the longitudinal axis of the main body. Again this enhances the compactness of the apparatus, and at the same time affords suitable access for switch means at the side of the apparatus. The switching components may be provided in any suitable configuration, for example a slide switch or push button switch.

The asymmetrically mounted vibrator may comprise a semi-circular element. Alternatively or additionally, it may comprise an asymmetrically mounted rotational cam, whose rotation results in a change in extent of its outward protrusion from its axis of rotation. This arrangement can provide a pulse that is transferred directly to a user through a flexible thin member.

Preferably, the vibration unit comprises a rotatable elongate axially mounted cam surface, the surface having a raised helical ridge to thereby provide a longitudinally moving contact ridge on rotation of the cam surface. The rotational cam surface conveniently comprises a cylinder on whose surface is provided said helical ridge. The rotational cam surface is conveniently provided adjacent a resilient contact strip, the helical ridge deforming the strip in the area where they are in contact. In this way, the user can experience a wave or stroking like sensation, due to longitudinal reciprocation of the ridge.

The vibration unit may be formed of a pair of opposing shell halves which are coupled together to enclose the unit's internal components in a water-resistant manner.

The present invention encompasses desensitising apparatus removably housing a vibration unit according to any of the preceding features.

The desensitising apparatus may comprise a user contact surface having an arcuate profile, for example a complex curve such as a hyperbolic paraboloid. This can optimise the contact surface area with the user's penis.

Preferably, the outer surface of the apparatus is profiled to promote a correct positioning for gripping the apparatus between a user's finger and palm. In this respect, the apparatus conveniently comprises side wings, one or each of which has a concave outer profile to accommodate a user's finger and/or the area at the palm at the connection with the finger. In this way, a user is guided intuitively how to hold the apparatus for correct and optimal performance.

The vibration unit preferably has variable vibration settings, to thereby apply different patterns, speeds and strengths of stimulation, for example according to a predetermined treatment regime. In this way, the level of stimulation can, for example, be stepped up, as the treatment regime advances over time. This can involve one or more of varying the speed of vibration, magnitude of pulse or varying the type of stimulation.

According to a further aspect of the present invention there is provided a desensitising system comprising:—a vibration unit as defined above and a plurality of desensitising apparatuses as defined above.

According to a further aspect of the present invention there is provided a method of using a desensitising apparatus, where the apparatus is profiled to be held in a user's palm by a user's bent finger; the user bringing the apparatus into contact with the underside of the glans of a penis by hooking their thumb around the back of the base of the glans and pivoting the apparatus into contact with the penis, using the contact between the thumb and the penis as a pivot surface; the user pivoting the apparatus about the pivot surface out of contact with the penis when stimulation by the apparatus is no longer required.

The user preferably pivots the apparatus out of contact with the penis when the urge to ejaculate is felt.

Conveniently, the pivoting into and out of contact actions are repeated for up to 45 minutes and at least 3 times a week for up to 6 weeks. More preferably, the pivoting into and out of contact steps are repeated for up to 30 minutes, at least 3 times a week for up to 6 weeks

Certain embodiments of the invention will now be described with reference to the attached drawings, of which:—

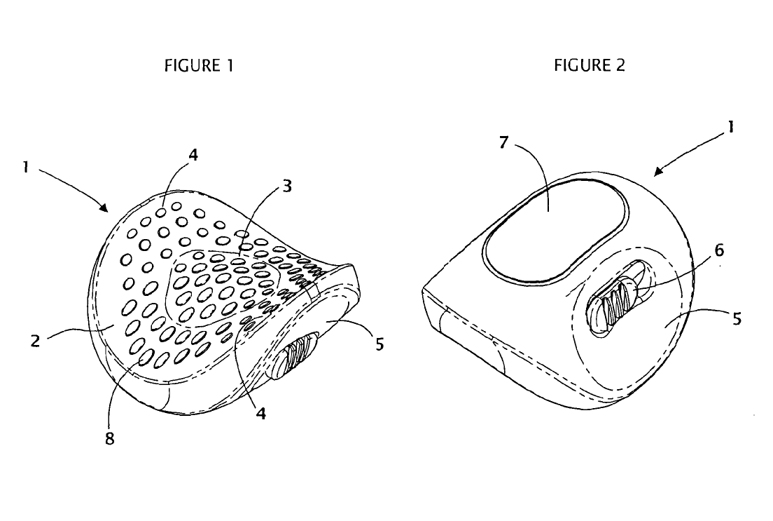

FIG. 1 shows a view from above of desensitising apparatus incorporating a first embodiment of the present invention;

FIG. 2 shows a view from below of desensitising apparatus incorporating a first embodiment of the present invention;

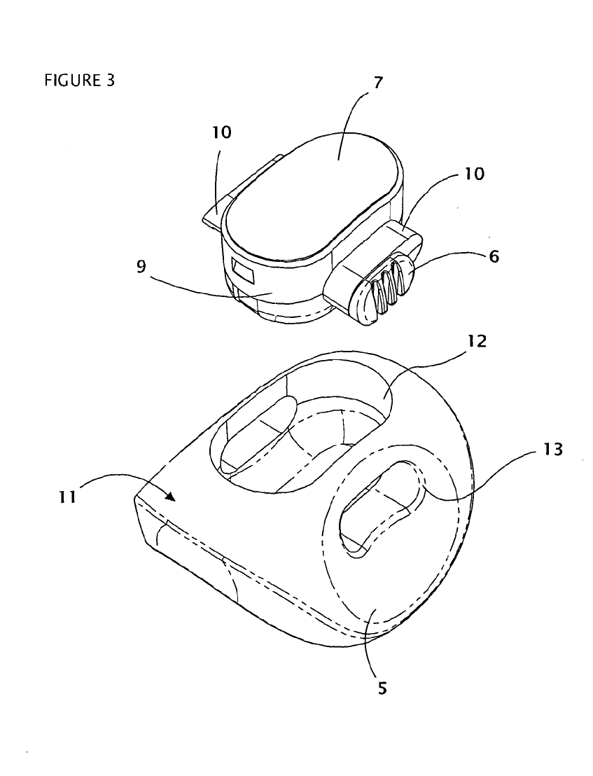

FIG. 3 shows vibration apparatus of the first embodiment when removed from the desensitising apparatus;

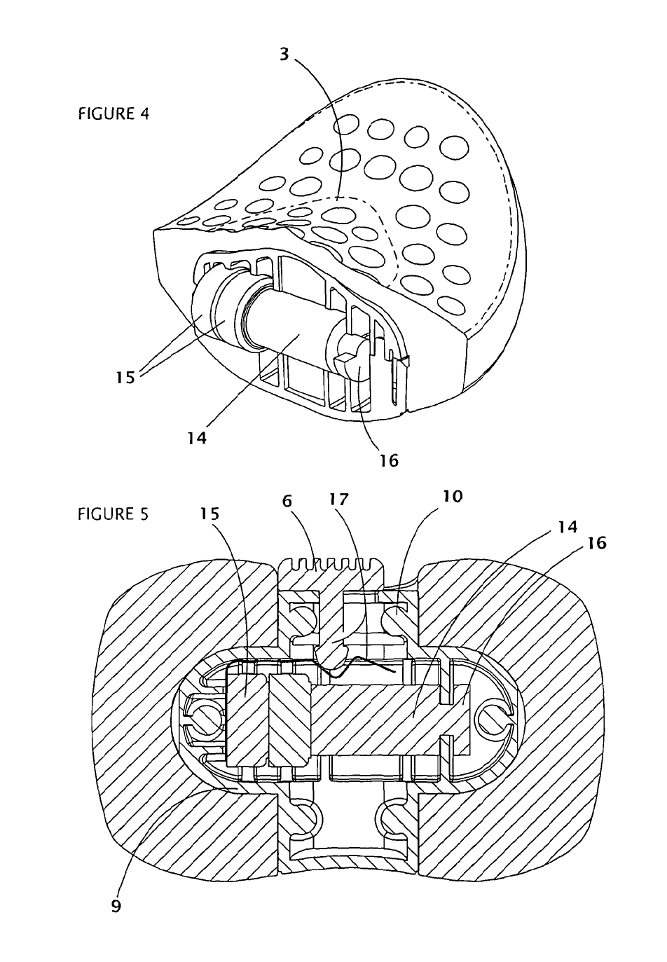

FIGS. 4 and 5 show cut away side and plan views of desensitising apparatus incorporating a first embodiment of the present invention;

FIG. 6 shows a cut away view of desensitising apparatus incorporating a second embodiment of the present invention;

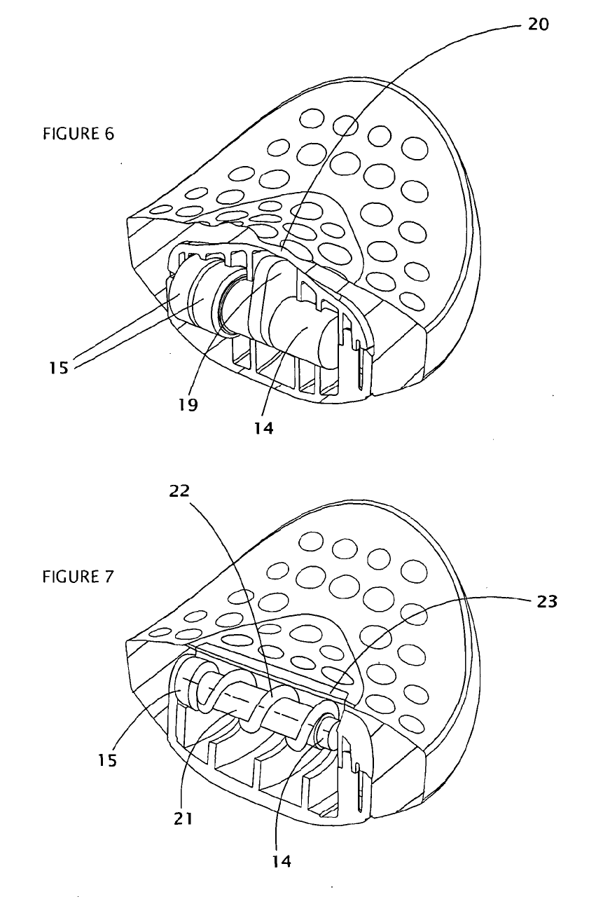

FIG. 7 shows a cut away view of desensitising apparatus incorporating a third embodiment of the present invention;

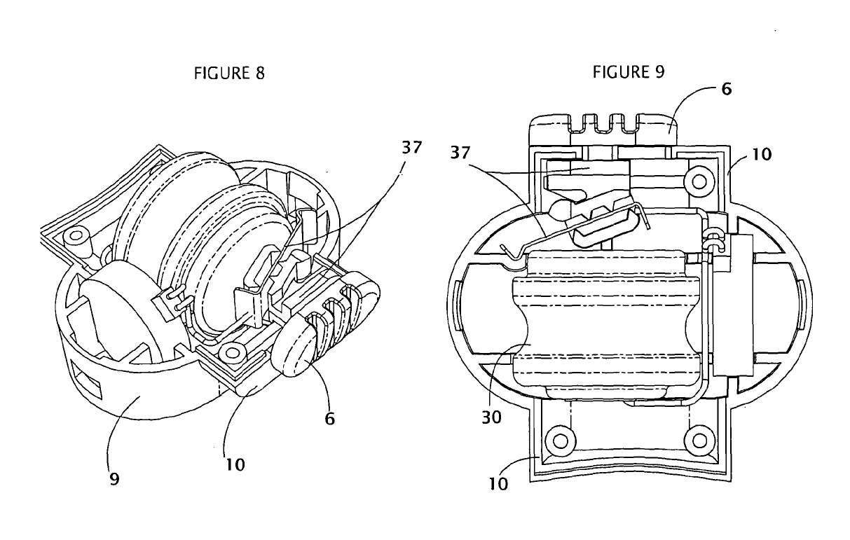

FIGS. 8 and 9 show cut away perspective and plan views of a fourth embodiment of the present invention; and

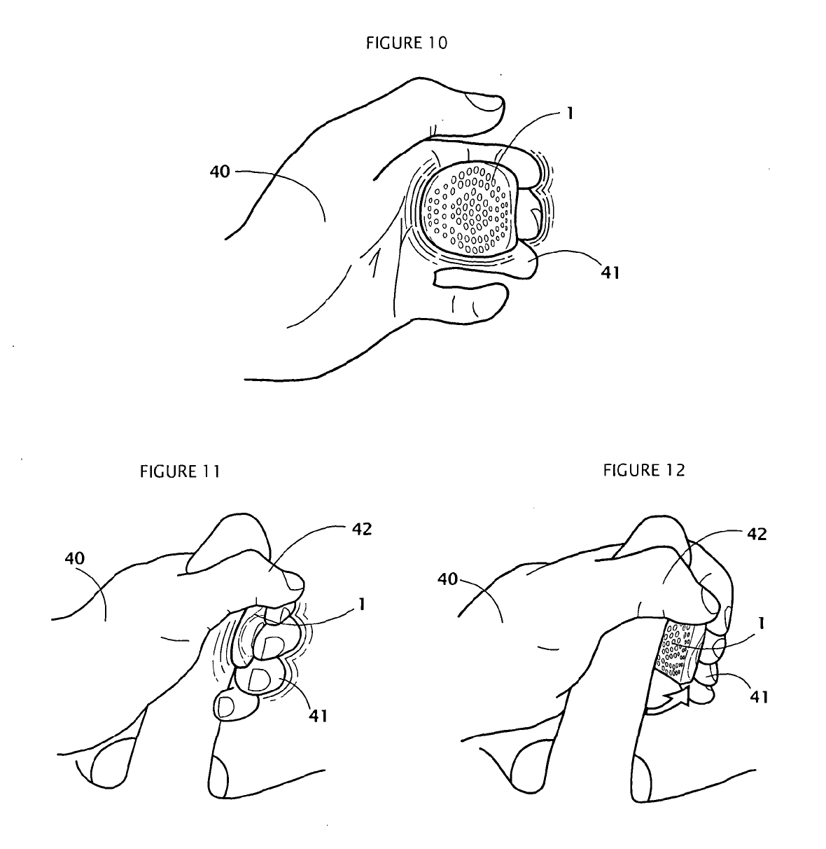

FIGS. 10, 11 and 12 show perspective views of an embodiment of the invention in use.

FIGS. 1 and 2 show views from above and below of desensitising apparatus 1 for stimulating the penis. The apparatus comprises a contact portion 2 having an open channel configuration. Such a channel like member will conform generally to the shape of the underside of the main body of the penis, particularly when erect or semi-erect. The contact portion is, in this regard, profiled to house and align the apparatus readily on a user's penis. This simplifies ensuring the apparatus is placed and held in the correct place during operation at the base of the glans.

On an inner surface of the channel like member there is provided a raised portion in the form of a dome 3. The dome extends inwardly of the channel-like member, namely inwardly of the profile of the underside of the main body of the penis. The dome 3 positively engages with the area at the base of the glans of the penis.

As a result of its three-dimensional profile, the dome significantly increases and thereby enhances the contact area with the penis at the base of the glans. Indeed, the dome promotes contact also with the corona on the underside of the penis. This enhanced contact augments the stimulating and hence desensitizing effect of the apparatus.

Side wings 4 are provided at the channel-like member. These side wings further promote the contact with the penis, particularly around the underside of the corona. As shown, the wings are contoured to extend around to match the profile of the corona, to thereby optimize the contact at this area of the penis.

As shown, the upper penis-facing surface takes the form of a complex curved surface. The complex curved surface has a first concave curve defining a channel for accommodating a length of a user's penis, at or adjacent the glans, in combination with a second convex curve forming the dome or hump 3 running transversely across the channel. The complex curve is in effect “saddle-shaped” or a hyperbolic paraboloid.

The unit has side portions 4 depending from the contact portion, which have opposed recessed portions for affording holding areas for a user of the apparatus. The holding areas are configured to allow the unit to be held comfortably and correctly by a user. A switch 6 for operating a vibration means 7 is provided within the recessed area.

Turning to the vibration unit 7, as shown in FIG. 3, this comprises a main body 9 and a pair of opposing side arm elements 10 projecting outwardly and normally therefrom. The vibration unit fits snugly within a resilient stimulation unit 11 of the desensitising apparatus. It is removably inserted by stretching and hence deforming the stimulation unit 11 to distort the aperture 12 thereby allowing the vibration unit to pass into the stimulation unit's interior. The side arm elements 10 are located in corresponding recesses within the stimulation unit, the exterior profile of the vibration unit thereby promoting anchoring thereof within the stimulation unit.

Referring to FIGS. 4 and 5, the vibration unit comprises a motor 14, a driven axle, batteries 15 and an asymmetrically mounted vibrator 16. The motor and driven axle are mounted along the longitudinal axis of the main body 9.

Switching components 17 are housed within the side arm elements 10. These components control the operation of the vibration unit. The side arm elements perform the function of an internal frame, affording the apparatus transverse rigidity, which enhances the ability of a user to grip the apparatus firmly in use.

The asymmetrically mounted vibrator 16 comprises a semi-circular element. Rotation of the asymmetrically mounted vibrator will lead to vibration of the vibration unit which is passed on to the stimulation unit which is in contact therewith.

As shown in FIG. 6, in a further embodiment of the present invention, the vibration unit comprises an asymmetrically mounted rotational cam 19, whose rotation results in a change in extent of its outward protrusion from its axis of rotation. In use the cam intentionally comes into intermittent contact with a flexible thin member 20 on an upper facing surface of the stimulation unit. In this way, as the cam 19 rotates, it generates a pulse in the member 20, which is thereby transferred directly to the user's penis when the apparatus is in place.

A further development of the present invention is shown in FIG. 7, where the vibration member comprises a rotatable cylinder 21 provided with a raised helical ridge 22 to thereby provide a longitudinally moving contact ridge on rotation of the cylinder. This moving ridge is passed on to the user through the flexible contact strip 23. The vibrating means may hence be controlled by a control means (not shown) to provide different vibration patterns to suit the user's requirements.

One such pattern may allow for an axial reciprocation of the ridge according to a range of vibrating speeds or a designated wave pattern.

The motor 14 may be provided concentrically with the cylinder 21 rather than axially adjacent as shown in FIG. 7.

The vibration unit is formed of a pair of opposing shell halves which are coupled together to enclose the unit's internal components in a water-resistant manner.

The present invention hence concerns apparatus for stimulating a penis to the extent that it becomes desensitized and tolerized, thereby delaying the latency period to ejaculation and hence may also be used for sufferers of premature ejaculation.

The present invention encapsulates a method of desensitizing a penis, using the apparatus, the method comprising using the apparatus on a repeated basis, to thereby tolerize the penis to stimulation.

In this respect, the vibration unit may have various vibration settings, to thereby apply different patterns, speeds and strengths of stimulation, for example according to a predetermined treatment regime. In this way, the level of stimulation can, for example, be stepped up, as the treatment regime advances over time. This can involve increasing the speed of vibration, magnitude of pulse or varying the type of stimulation.

In view of its removable nature, the vibration unit can moreover be interchanged between different types of stimulation apparatus, having for example different stimulation projection patterns. In one treatment regime, a user may be provided with a vibration unit and a number of stimulation apparatuses, with instructions on when and how long to use each.

In the above embodiments, the vibration unit comprises a cam motor arrangement. However, a flat cam-less “pancake” motor may also be used as shown in FIGS. 8 and 9. In this embodiment, a “pancake” motor 30 provides the vibratory motion. Switching components 37 are coupled with switch 6 for operational control of the vibratory effect.

In use of the device, a user first applies lubricant generously to the under surface of the glans of the penis. The user then turns on the vibration unit using the switch 6 to start the device vibrating.

The device is then rested in the user's hand 40 with the concave studded area facing upwards in the cupped fingers 41 as shown in FIG. 10. The user's thumb 42 is then hooked around the back of the base of the glans as shown in FIG. 11 with the vibrating device coming into contact with the under surface of the glans. The user can masturbate gently with the device in place. This corresponds to the Start element of the Start/Stop technique.

Once the urge to ejaculate is felt, the device is released from contact by distancing the cupped fingers/palm from the under surface of the penis whilst maintaining the hooked thumb around the back of the glans to act as a pivot as shown in FIG. 12. This corresponds to the Stop element of the Start/Stop technique. The exterior profile of the device facilitates holding in the cupped fingers and pivoting about the back of the glans. In this respect the side wings are profiled to accommodate a users's middle finger and the slightly raised area at the base of the finger where it connects to the palm. As shown in FIGS. 1,2 and 10, the side wings have concave areas 5 for this purpose. Affording such a profile guides a user to intuitively hold the apparatus in the correct position in their hand.

This exercise is repeated until the user reaches the point of no return. The technique should be used for up to 30 minutes, sometimes longer, say up to 45 minutes, if needed depending upon response and at least 3 times a week. The training programme should continue for up to 6 weeks.

1. A vibration unit for use in penis desensitising apparatus, the vibration unit comprising a main body and one or more side elements projecting outwardly therefrom, the vibration unit thereby having an exterior profile for promoting anchoring thereof within the desensitising apparatus.

2. The vibration unit of claim 1, wherein the vibration unit main body is substantially elongate, with the one or more side elements extending substantially normally therefrom.

3. The vibration unit of claim 1, wherein the vibration unit is removably housed within a desensitising apparatus, which itself is formed of a flexible resilient material.

4. The vibration unit of claim 3, wherein the desensitising apparatus is formed with a deformable aperture which in its undeformed state matches at least part of the exterior profile of the vibration unit.

5. The vibration unit of claim 1, wherein the vibration unit comprises a motor, a driven axle, and an asymmetrically mounted vibrator.

6. The vibration unit of claim 5, wherein the motor and driven axle are mounted along the longitudinal axis of the main body.

7. The vibration unit of claim 5, wherein switching components are housed within the one or more side elements which are disposed normally to the longitudinal axis of the main body.

8. The vibration unit of claim 5, wherein the asymmetrically mounted vibrator comprises a semi-circular element.

9. The vibration unit of claim 1, wherein the vibration unit comprises an asymmetrically mounted rotational cam, whose rotation results in a change in extent of its outward protrusion from its axis of rotation.

10. The vibration unit of claim 1, wherein the vibration unit comprises a rotatable elongate axially mounted cam surface, the surface having a raised helical ridge to thereby provide a longitudinally moving contact ridge on rotation of the cam surface.

11. The vibration unit of claim 10, wherein the rotational cam surface comprises a cylinder on whose surface is provided said helical ridge.

12. The vibration unit of claim 10, wherein the rotational cam surface is provided adjacent a resilient contact strip, the helical ridge deforming the strip in the area where they are in contact.

13. The vibration unit of claim 1, wherein the vibration unit is formed of a pair of opposing shell halves which are coupled together to enclose the unit's internal components in a water-resistant manner.

14. A desensitising apparatus removably housing the vibration unit of claim 1.

15. The desensitizing apparatus of claim 14, comprising a user contact surface having an arcuate profile.

16. The desensitizing apparatus of claim 14, wherein the user contact surface profile is a complex curve.

17. The desensitizing apparatus of claim 14, wherein the user contact surface is a hyperbolic paraboloid.

18. The desensitizing apparatus of claim 14, wherein the vibration unit has variable vibration settings, to thereby apply one or more of different patterns, speeds and strengths of stimulation.

19. The desensitizing apparatus of claim 18, wherein the vibration settings are varied according to a predetermined treatment regime.

20. The desensitizing apparatus of claim 14, wherein an outer surface of the apparatus is profiled to promote a correct positioning for gripping the apparatus between a user's fingers and palm.

21. The desensitizing apparatus of claim 20, wherein the apparatus comprises side wings, one or each of which has a concave outer profile to accommodate a user's finger and/or the area at the palm at the connection with the finger.

22. A desensitising system comprising
the vibration unit of claim 1, and a plurality of desensitising apparatuses removably housing the vibration unit of claim 1.

23. A method of using a desensitising apparatus, where the apparatus is profiled to be held in a user's palm by a user's bent finger;
the user bringing the apparatus into contact with the underside of the glans of a penis by hooking their thumb around the back of the base of the glans and pivoting the apparatus into contact with the penis, using the contact between the thumb and the penis as a pivot surface;
the user pivoting the apparatus about the pivot surface out of contact with the penis when stimulation by the apparatus is no longer required.

24. The method of claim 23, wherein the user pivots the apparatus out of contact with the penis when the urge to ejaculate is felt.

25. The method of claim 23, wherein the pivoting into and out of contact actions are repeated for up to 45 minutes and at least 3 times a week for up to 6 weeks.

26. The method of claim 23, wherein the pivoting into and out of contact steps are repeated for up to 30 minutes.
